SEQUENCE LISTING (16 sequences)

SEQ ID NO: 1 - protein, 4 aa, artificial sequence
  SOURCE 1..4: /mol_type=“protein” /note=“Alanin-Lysin-Prolin-Serin” /organism=“artificial sequence synthetic
AKPS

SEQ ID NO: 2 - protein, 4 aa, artificial sequence
  SOURCE 1..4: /mol_type=”protein“ /note=”Lysin-Prolin-Serin-Tyrosin“ /organism=”artificial sequence“ synthetic
KPSY

SEQ ID NO: 3 - protein, 4 aa, artificial sequence
  SOURCE 1..4: /mol_type=”protein“ /note=”Prolin-Serin-Tyrosin-Prolin“ /organism=”artificial sequence“ synthetic
PSYP

SEQ ID NO: 4 - protein, 4 aa, artificial sequence
  SOURCE 1..4: /mol_type=”protein“ /note=”Serin-Tyrosin-Prolin-Prolin“ /organism=”artificial sequence“ synthetic
SYPP

SEQ ID NO: 5 - protein, 4 aa, artificial sequence
  SOURCE 1..4: /mol_type=”protein“ /note=”Tyrosin-Prolin-Prolin-Threonin“ /organism=”artificial sequence“ synthetic
YPPT

SEQ ID NO: 6 - protein, 5 aa, artificial sequence
  SOURCE 1..5: /mol_type=”protein“ /note=”Alanin-Lysin-Prolin-Serin-Tyrosin“ /organism=”artificial sequence“ synthetic
AKPSY

SEQ ID NO: 7 - protein, 5 aa, artificial sequence
  SOURCE 1..5: /mol_type=”protein“ /note=”Lysin-Prolin-Serin-Tyrosin-Prolin“ /organism=”artificial sequence“ synthetic
KPSYP

SEQ ID NO: 8 - protein, 5 aa, artificial sequence
  SOURCE 1..5: /mol_type=”protein“ /note=”Prolin-Serin-Tyrosin-Prolin-Prolin“ /organism=”artificial sequence“ synthetic
PSYPP

SEQ ID NO: 9 - protein, 5 aa, artificial sequence
  SOURCE 1..5: /mol_type=”protein“ /note=”Serin-Tyrosin-Prolin-Prolin-Threonin“ /organism=”artificial sequence“ syntehtic
SYPPT

SEQ ID NO: 10 - protein, 6 aa, artificial sequence
  SOURCE 1..6: /mol_type=”protein“ /note=”Alanin-Lysin-Prolin-Serin-Tyrosin-Prolin“ /organism=”artificial sequence“ synethic
AKPSYP

SEQ ID NO: 11 - protein, 6 aa, artificial sequence
  SOURCE 1..6: /mol_type=”protein“ /note=”Lysin-Prolin-Serin-Tyrosin-Prolin-Prolin“ /organism=”artificial sequence“ synthetic
KPSYPP

SEQ ID NO: 12 - protein, 6 aa, artificial sequence
  SOURCE 1..6: /mol_type=”protein“ /note=”Prolin-Serin-Tyrosin-Prolin-Prolin-Threonin“ /organism=”artificial sequence“ synthetic
PSYPPT

SEQ ID NO: 13 - protein, 7 aa, artificial sequence
  SOURCE 1..7: /mol_type=”protein“ /note=”Alanin-Lysin-Prolin-Serin-Tyrosin-Prolin-Prolin“ /organism=”artificial sequence“ synthetic
AKPSYPP

SEQ ID NO: 14 - protein, 7 aa, artificial sequence
  SOURCE 1..7: /mol_type=”protein“ /note=”Lysin-Prolin-Serin-Tyrosin-Prolin-Prolin-Threonin“ /organism=”artificial sequence“ synthetic
KPSYPPT

SEQ ID NO: 15 - protein, 8 aa, artificial sequence
  SOURCE 1..8: /mol_type=”protein“ /note=”Alanin-Lysin-Prolin-Serin-Tyrosin-Prolin-Prolin-Threonin“ /organism=”artificial sequence“ synthetic
AKPSYPPT

SEQ ID NO: 16 - protein, 11 aa, artificial sequence
  SOURCE 1..11: /mol_type=”protein“ /note=”sequence is a copound of formula (IV);Cystein- Alanin-Lysin-Prolin-Serin-Tyrosin-Prolin-Prolin-Threonin-LDOPA-Ly sin“ /organism=”artificial sequence“ synthetic
CAKPSYPPTXK

(19) United States
(12) Patent Application Publication
(10) Pub. No.: US 2016/0130484 A1
(43) Pub. Date: May 12, 2016

(54) MULTI-COMPONENT ADHESIVE FOR PRODUCING AN ADHESIVE HYDROGEL

(71) Applicants: FRAUNHOFER-GESELLSCHAFT ZUR FÖRDERUNG DER ANGEWANDTEN FORSCHUNG E.V., München (DE); TECHNISCHE UNIVERSITÄT DARMSTADT, Darmstadt (DE); JOHANN WOLFGANG GOETHE-UNIVERSITÄT, Frankfurt am Main (DE)

(72) Inventors: Klaus Rischka, Tostedt (DE); Robert Sader, Frankfurt (DE); Hendrik Bargel, Bayreuth (DE); Maria Kozielec, Bremen (DE)

(21) Appl. No.: 14/769,299

(22) PCT Filed: Feb. 21, 2014
(86) PCT No.: PCT/EP2014/053469

§ 371 Date: Aug. 20, 2015

(30) Foreign Application Priority Data
Feb. 21, 2013 (DE) 10 2013 202 874.1

(51) Int. Cl. C09J 171/02; A61L 24/00; A61L 24/06

(52) U.S. Cl. CPC C09J 171/02; A61L 24/06; A61L 24/0031; A61L 2430/12

(57) ABSTRACT

The present invention primarily relates to a multicomponent adhesive for producing an adhesive hydrogel, comprising the compound defined below of the formula (I) and one, two, three, four or more further constituents. The invention further relates to the use of said multicomponent adhesive and corresponding methods for gluing two surfaces together. The present invention further relates to a method for producing a corresponding adhesive hydrogel. The invention also relates to a kit comprising a corresponding multicomponent adhesive and one or more further constituents. The invention also relates to articles which comprise a multicomponent adhesive according to the invention or an adhesive hydrogel produced therefrom.

The present invention primarily relates to a multicomponent adhesive for producing an adhesive hydrogel, comprising the compound defined below of the formula (I) and one, two, three, four or more further constituents. The invention further relates to the use of said multicomponent adhesive and corresponding methods for gluing two surfaces together. The present invention further relates to a method for producing a corresponding adhesive hydrogel. The invention also relates to a kit comprising a corresponding multicomponent adhesive and one or more further constituents. The invention also relates to articles which comprise a multicomponent adhesive according to the invention or an adhesive hydrogel produced therefrom.

Further aspects of the present invention follow from the following description and in particular from the appended patent claims.

In modern surgery, there is a constant need for new biocompatible adhesives for gluing tissues. An important field of application for such biocompatible adhesives lies in the field of dental implantology. In the use of implants, in spite of careful procedure on the part of the (dental) surgeon, there is always the danger that bacteria will penetrate into the interface between implant and the tissue surrounding the implant (e.g. peri-implant mucosa) and lead to infections. However, infections of the peri-implant mucous membrane (mucosa) without involvement of bone are reversible and are described as mucositis. With advancing inflammation with progressive, irreversible bone loss, this is referred to as periimplantitis. Because of the bone loss in perimplantitis, this leads to implant loosening and can ultimately also lead to implant loss. At present, both mucositis and also perimplantitis can only be avoided by regular checks and appropriate therapeutic measures after the diagnosis of the first signs of mucositis. This is associated with major treatment effort for the treating (dental) surgeon and patient and with high costs.

Stabilization or sealing of the mucosa and tissue layers on the implant body, which is achieved by the use of biocompatible adhesives, not only results in a greatly reduced danger of mucositis or periimplantitis, but also decreases the healing time of the implants on the tissue surrounding the implant (e.g. periimplantal supporting tissue). An important prerequisite for successful implantation is that the implant has adequate primary stability after introduction into the bone. This means that it must not sit loose directly after introduction, but is absolutely, i.e. to the maximum extent possible, anchored firmly in the bone. This primary stability is supported by appropriate gluing. On account of the stabilization, chewing forces acting on the implant-bone structure, which is in construction and conversion after the insertion of the implant, do not act to a decisive extent on newly formed tissue, so that this is not destroyed again. Through stabilization or sealing of the implant on the tissue surrounding the implant, the oral reconstruction in the long term has a very good starting situation for complete rehabilitation. As a result, the currently usual effort for treatment of peri-implant supporting tissue invasion and the costs associated therewith can be reduced or completely avoided. Compared to the implant lifetimes of previous treatment methods without biocompatible adhesives, the use of biocompatible adhesives also leads to markedly longer implant survival times.

As well as the use of biocompatible adhesives, further surgical, therapeutic and other applications are also possible in implantology. Thus for example cuts can be glued by the application of appropriate adhesives. Even gluing of bone, cartilage and or soft tissue is possible.

Thus for example, immobilization of bone fractures can be achieved, with which the subsequent healing process can take place markedly more rapidly. In this, the intrinsic load-bearing capacity of the bone tissue gradually increases and the biocompatible adhesive is gradually degraded until only pure cell tissue or bone material is still present.

In the literature, many adhesives and sealing materials which are suitable for medical use are disclosed.

EP 1 609 431 B1 discloses a cell-occluding membrane which is obtained by reaction of at least two precursor molecules. The cell-occluding membrane described is not an adhesive, but the membrane can also be used in bone regeneration or tissue regeneration.

In Bubaker et al., Biomaterials 31 (2010) 420, a medical adhesive is disclosed. The adhesive described can be converted into a hydrogel by oxidation with sodium periodate. The gluing of fatty tissue and liver tissue in mice is also described.

Bruce Lee et al., Macromolecules 39 (2006) 1740 discloses an adhesive which can be converted into a gel by light curing. The adhesive and the gel resulting therefrom have a high content of 3,4-dihydroxyphenylalanine (DOPA). As a possible application example for the adhesives disclosed or the gels formed therefrom, the use as a bioadhesive is described.

WO 2012/009664 A2 discloses medical adhesives and antifouling coatings based on proteins of the natural marine mussel adhesives.

The purpose of the present invention was to provide an adhesive which after hardening (setting) has at least one, but preferably several, of the following properties:
good biocompatibility
little or no toxicity
biodegradability
ability to absorb and/or to incorporate active substances and optionally capability for delayed (re-)release of absorbed or incorporated active substances
no immune reaction in the animal and/or human body
good adhesion to biological tissue of allogenic, heterogenic or synthetic origin and/or to medical implants
good barrier action against bacteria.

This problem is solved by a multicomponent adhesive for producing an adhesive hydrogel, comprising
a) a compound of the formula (I)

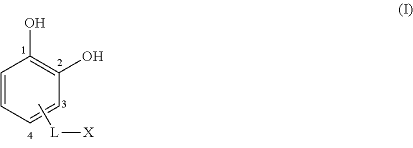

wherein
X is selected from the group consisting of SH, NH₂ and alpha,beta-unsaturated carbonyl groups and
L is a linker which links the 1,2-dihydroxyphenyl unit with X, wherein the linker L is linked with the 1,2-dihydroxyphenyl unit in the 3 or 4 position,
and
b) one, two, three, four or more compounds,
   each containing one, two, three, four, five, six, seven, eight or more structural units (II)

B-L′-Y (II)
wherein in each of the structural units (II) independently of the meaning in other structural units (II)
B is a linkage point,
L′ is a linker and
Y is selected from the group consisting of SH, NH₂ and alpha,beta-unsaturated carbonyl groups and can be reacted with the group X with formation of at least one covalent bond,
wherein
(i) one, two, three, four or more of the compounds which each contain one, two, three, four, five, six, seven, eight or more structural units (II), each additionally comprise a residue Z, which independently of every other residue Z of another compound is selected from the group consisting of SH, NH₂ and alpha,beta-unsaturated carbonyl groups and can be reacted with a group Y with formation of at least one covalent bond
   and/or
(ii) the multicomponent adhesive additionally comprises one, two, three, four or more compounds, which independently of one another each comprise two, three, four, five, six, seven, eight or more structural units (III)

B′-L″-Z′ (III)
wherein in each of the structural units (III) independently of the meaning in other structural units (III)
B′ is a linkage point,
L″ is a linker and
Z′ is selected from the group consisting of SH, NH₂ and alpha,beta-unsaturated carbonyl groups and can be reacted with a group Y with formation of at least one covalent bond.

In the context of this text, an adhesive is understood to be a substance mixture which by chemical reaction of its constituents and/or by the action of external substances sets and can thereby bind materials and/or tissue by surface adhesion such that the bond has adequate internal strength (cohesion).

A “multicomponent adhesive” is generally understood to mean an adhesive which consists of two or more reactive components spatially separated from one another, which are mixed for the reaction and if necessary still further treated. The constituents (e.g. a compound of the formula (I)) of the multicomponent adhesive according to the invention are thus divided into two, three or more separate components, wherein preferably components, in which X, Y, Z or Z′ are SH and/or NH₂, are separated from components, in which X, Y, Z or Z′ is an alpha,beta-unsaturated carbonyl group. In certain embodiments of multicomponent adhesives according to the invention, e.g. when a separation of constituents intended for reaction with one another, in which X, Y, Z or Z′ are SH and/or NH₂, from constituents in which X, Y, Z or Z′ is an alpha,beta-unsaturated carbonyl group, is not possible or useful, the further constituents of the multicomponent adhesive are divided such that nonetheless formation of the adhesive hydrogel (by setting) does not occur before the mixing of the components. This can for example come about because the pH of individual or of all components of the multicomponent adhesive is adjusted such that formation of the adhesive hydrogel only takes place after the mixing of the components or after the application of the multicomponent adhesive in an appropriate environment which has an appropriate pH (e.g. oral cavity).

In the context of this text, “hydrogels” are understood to be gels based on hydrophilic, but water-insoluble polymers which are present as three-dimensional networks. Through the hydrophilic nature of the three-dimensional networks, hydrogels are capable of absorbing water. Preferably the hydrogels contain water. However, in the context of this text, dried hydrogels, which contain no water or only traces thereof, are also understood to be hydrogels provided that they are capable of absorbing (additional) water.

An “adhesive hydrogel” is understood to be a hydrogel which both displays the properties of a hydrogel and also can bind materials and/or tissue by surface adhesion such that the bond has adequate internal strength (cohesion). After the mixing of the components of a multicomponent adhesive according to the invention, an adhesive hydrogel is formed by chemical reaction of the constituents of the individual components.

According to a first preferred configuration of the present invention, a multicomponent adhesive according to the invention for producing an adhesive hydrogel comprises
a) a compound of the formula (I)

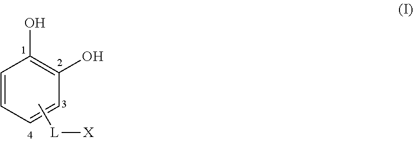

wherein
X is selected from the group consisting of SH, NH₂ and alpha,beta-unsaturated carbonyl groups and
L is a linker which links the 1,2-dihydroxyphenyl unit with X, wherein the linker L is linked with the 1,2-dihydroxyphenyl unit in the 3 or 4 position,
and
b) one, two, three, four or more compounds,
   each containing one, two, three, four, five, six, seven, eight or more structural units (II)

B-L′-Y (II)
wherein in each of the structural units (II) independently of the meaning in other structural units (II)
B is a linkage point,
L′ is a linker and
Y is selected from the group consisting of SH, NH₂ and alpha,beta-unsaturated carbonyl groups and can be reacted with the group X with formation of at least one covalent bond,
wherein
(i) one, two, three, four or more of the compounds which each contain one, two, three, four, five, six, seven, eight or more structural units (II), each additionally comprise a residue Z, which independently of every other residue Z of another compound is selected from the group consisting of SH, NH₂ and alpha,beta-unsaturated carbonyl groups and can be reacted with a group Y with formation of at least one covalent bond.

According to a second, equally preferred configuration of the present invention, a multicomponent adhesive according to the invention for producing an adhesive hydrogel comprises
a) a compound of the formula (I)

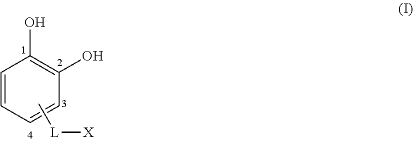

wherein
X is selected from the group consisting of SH, NH₂ and alpha,beta-unsaturated carbonyl groups and
L is a linker which links the 1,2-dihydroxyphenyl unit with X, wherein the linker L is linked with the 1,2-dihydroxyphenyl unit in the 3 or 4 position,
and
b) one, two, three, four or more compounds,
   each containing one, two, three, four, five, six, seven, eight or more structural units (II)

B-L′-Y (II)
wherein in each of the structural units (II) independently of the meaning in other structural units (II)
B is a linkage point,
L′ is a linker and
Y is selected from the group consisting of SH, NH₂ and alpha,beta-unsaturated carbonyl groups and can be reacted with the group X with formation of at least one covalent bond,
   and
(ii) additionally one, two, three, four or more compounds, which independently of one another each comprise two, three, four, five, six, seven, eight or more structural units (III)

B′-L″-Z′ (III)
wherein in each of the structural units (III) independently of the meaning in other structural units (III)
B′ is a linkage point,
L″ is a linker and
Z′ is selected from the group consisting of SH, NH₂ and alpha,beta-unsaturated carbonyl groups and can be reacted with a group Y with formation of at least one covalent bond.

It has been found that multicomponent adhesives according to the invention display especially good properties when at least one component of the multicomponent adhesive has a specific pH. After mixing of the components for the purpose of forming an adhesive hydrogel, in particular with the preferred presence of SH groups (Y, X and/or Z′ then mean SH in the particular corresponding compound or structural unit of the formula (I), (II) or (III)) at a pH of >8, side reactions can occur, which can adversely affect the production of the adhesive hydrogel. At a pH of <6, the reaction rate of the reaction mixture obtained by mixing of the components in the formation of the adhesive hydrogel is often lowered, so that the formation of the adhesive hydrogel is retarded. A multicomponent adhesive according to the invention in which one, two, three or all components have a pH in the range from 6 to 8, preferably in the range from 7 to 8, especially preferably in the range from 7.5 to 7.8, is therefore preferred.

Preferably the components of the multicomponent adhesive according to the invention are configured such that so after the mixing of all the components, a pH in the range from 6 to 8, preferably in the range from 7 to 8, especially preferably in the range from 7.5 to 7.8 is present in the resulting reaction mixture. This applies in particular with the preferred presence of SH groups (Y, X and/or Z′ then mean SH in the particular corresponding compound or structural unit of the formula (I), (II) or (III)).

Such a pH range is physiologically very well tolerated and is for example usually present in the oral cavity. Consequently, the preferred multicomponent adhesives according to the invention exhibit especially good biocompatibility. Moreover the formation of an adhesive hydrogel proceeds especially well in this pH range, in particular with the preferred presence of SH groups.

In some embodiments it has been found advantageous, to stabilize the pH range or value of the reaction mixture resulting from a multicomponent adhesive according to the invention by addition of a buffer system. A multicomponent adhesive according to the invention comprising c) a buffer consisting of a buffer acid and a corresponding buffer base, where the pK_{B} of the buffer base is not less than 2, preferably is not less than 4, is therefore preferred.

The use of a buffer system leads to a stabilization of the pH range or value in the reaction mixture of a multicomponent adhesive according to the invention. Thereby for example possible side reactions which otherwise can arise during the formation of an adhesive hydrogel at pH values of >8 can be avoided. This applies in particular with the preferred presence of SH groups (Y, X and/or Z′ then mean SH in the particular corresponding compound or structural unit of the formula (I), (II) or (III)). Moreover, through use of a buffer system the pH values of the components and the pH of the mixture resulting through mixing of all components can be very precisely adjusted.

Preferred in particular is a multicomponent adhesive according to the invention, in which the molar ratio of buffer acid to buffer base lies in the range from 1:10 to 10:1, preferably in the range from 1:8 to 8:1, especially preferably in the range from 1:5 to 5:1. In this range of the molar ratio of buffer acid to the buffer base, the buffer always has outstanding buffering capacity.

Many buffer systems which can be adjusted to a desired pH in the range between 6 and 8 are known to those skilled in the art.

In our own studies, it has been found that buffer systems which contain primary amine groups and/or buffer systems which contain 3-(N-morpholino)propanesulfonic acid (MOPS) in many cases impede the formation of an adhesive hydrogel according to the invention from the multicomponent adhesive according to the invention to a significant extent; the presence of sulfonic acid groups appears on the whole not to be preferred. Probably side reactions take place between the amine groups and/or sulfonic acid groups of the buffer and the further components of the multicomponent adhesive intended for reaction with one another. Accordingly, a multicomponent adhesive according to the invention, in which the buffer acid and/or buffer base enter into no chemical reactions with the components of the multicomponent adhesive interfering with the formation of an adhesive hydrogel is especially preferred. In particular, a multicomponent adhesive according to the invention in which the buffer acid and/or buffer base contains no amine groups and/or sulfonic acid groups is especially preferred.

In our own studies, many buffer systems could be identified whose pH can be adjusted in the range between 6 and 8 and which enter into no undesired interactions (e.g. chemical reactions) with the constituents of the multicomponent adhesive according to the invention. Hence a multicomponent adhesive according to the invention, comprising one or more buffers (buffer systems) selected from the group consisting of maleate buffer, citrate buffer, bis(2-hydroxyethyl)amino-tris(hydroxymethyl)methane buffer (bis-tris buffer), phosphate buffer, N-(2-acetamido)-iminodiacetic acid (ADA), carbonate buffer, imidazole buffer, 1,3-bis[tris-(hydroxymethyl)-methylamino]propane buffer (bis-tris-propane buffer), triethanolamine buffer, tricin buffer and bicin buffer is preferred.

The formation of an adhesive hydrogel according to the invention takes place by chemical reaction of the constituents of the multicomponent adhesive according to the invention. In this, the SH— and/or NH₂ groups preferably react with the alpha,beta-unsaturated carbonyl groups in the context of a Michael addition. Here the SH— and/or NH₂ groups act as Michael donors and the alpha,beta-unsaturated carbonyl groups as Michael acceptors. This Michael addition proceeds especially well in the presence of a strong base. Hence a multicomponent adhesive according to the invention which comprises as component d) one or more bases with a pK_{B}<1 is preferred.

Especially preferred is such a multicomponent adhesive according to the invention, in which one or the bases with a pK_{B}<1 is ammonia, ammonium hydroxide and/or hydroxides of the alkali metals or alkaline earth metals, preferably sodium hydroxide or potassium hydroxide. The resulting multicomponent adhesives according to the invention display very good biocompatibility.

### Constituent a), Compound of the Formula (I):

The compound of the formula (I) in a multicomponent adhesive according to the invention is jointly responsible for outstanding adhesion of the adhesive hydrogel resulting from the multicomponent adhesive according to the invention. The 1,2-dihydroxyphenyl unit of the compound of the formula (I) is capable of developing strong adhesive forces to other surfaces (e.g. biological tissues or titanium surfaces). In our own studies, it has been found that the linker L in the compound of the formula (I) has an influence on the properties of the multicomponent adhesive or the adhesive hydrogel resulting therefrom (e.g. biocompatibility, biodegradability, good adhesion to biological tissue of allogenic, heterogenic or synthetic origin and/or to medical implants).

According to a preferred configuration of the multicomponent adhesive according to the invention, the linker L of the compound of the formula (I) is an organic molecule unit, the chain members whereof consist of (a) exclusively carbon atoms or (b) carbon atoms and hetero atoms. As a result of this, the multicomponent adhesive according to the invention or the adhesive hydrogel according to the invention resulting therefrom displays good biocompatibility, biodegradability and/or little or no toxicity.

In certain embodiments of the multicomponent adhesive according to the invention, it has been found advantageous to provide the linker L in a defined length. A multicomponent adhesive according to the invention, wherein the number of chain members of the linker L lies in the range from 5 to 65, preferably 6 to 60, preferably 24 to 36 is preferred. It has been found in our own studies that the adhesive hydrogels resulting from such multicomponent adhesives according to the invention display good adhesion to biological tissue of allogenic, heterogenic or synthetic origin and/or to medical implants.

In the context of the present text, a chain member of the linker L is understood to mean a single atom (chain atom or chain member) which in combination with further atoms consecutively linked by covalent bonds (further chain atoms or chain members) links the 1,2-dihydroxyphenyl unit of the compound of the formula (I) by the shortest route with the functional group X. Only the atoms which lie on the shortest route between the 1,2-dihydroxyphenyl unit of the compound of the formula (I) and X are counted as chain members; atoms in side chains are not considered. For the case where a compound of the formula (I) comprises both one or more SH groups and also one or more NH₂ groups only the SH group(s) is (are) considered. For the case where a compound of the formula (I) comprises two or more SH groups or (in the absence of SH groups) NH₂ groups, the group SH or NH₂ which is linked by the shortest route with the 1,2-dihydroxyphenyl unit and the SH or NH₂ group is regarded as X. For example, the number of the chain members of the linker L in the following compound of the formula (IV) is 30 (this is the number of the atoms between the group SH and the 1,2-dihydroxyphenyl unit).

The linker L here can be configured in a great variety of ways. Especially preferred however is a multicomponent adhesive according to the invention in which the linker L is a hydrocarbon, a polyether or a peptide or comprises such a group. The adhesive hydrogels resulting from such multicomponent adhesives according to the invention display very good biocompatibility.

In order to improve the bonding (adhesion) of the adhesive hydrogel produced from the multicomponent adhesive according to the invention, it has been found advantageous if alkylamines are also present in the compound of the formula (I) as well as the 1,2-dihydroxyphenyl unit. Accordingly, a multicomponent adhesive according to the invention wherein the linker L contains one, two, three, four or more alkylamines as side chain is preferred.

Likewise preferred is a multicomponent adhesive according to the invention, wherein the linker L is or comprises a peptide, wherein the peptide comprises at least 2 N atoms in the chain linking X with the 1,2-dihydroxyphenyl unit, preferably 2 to 20, more preferably 5 to 15, especially preferably 8 to 12 N atoms in the chain linking X with the 1,2-dihydroxyphenyl unit. In this, only N atoms which are chain members are counted. For example, the number of the N atoms in the chain linking X with the 1,2-dihydroxyphenyl unit in the following compound of the formula (IV) is 9. With use of peptides in or as linkers L, multicomponent adhesives according to the invention or the adhesive hydrogel according to the invention resulting therefrom display especially good biocompatibility and/or biodegradability.

Also preferred according to the invention is a multicomponent adhesive, wherein the compound of the formula (I) contains one or more structural units (IX)

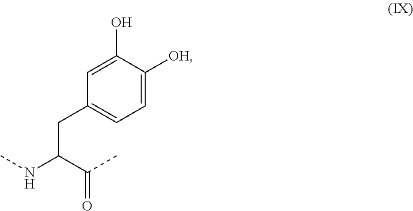

wherein at least one of the dashed lines represents a covalent bond to further structural units. For the case where the structural unit (IX) is present terminally in a compound of the formula (I), one of the dashed lines corresponds to hydrogen or —OH, depending on whether the structural units (IX) are covalently bound with further structural units via the carboxylic acid function or via the amine function.

In some applications, a multicomponent adhesive according to the invention in which the compound of the formula (I) contains specific amino acid units or combinations of specific amino acid units has been found particularly suitable. Especially preferable here is a multicomponent adhesive according to the invention wherein the compound of the formula (I) contains at least one cysteine unit, preferably one L-cysteine unit (formula X).

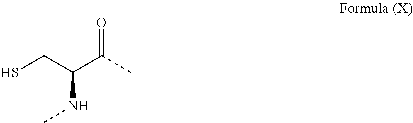

At least one of the dashed lines in formula (X) represents a covalent bond to further structural units. For the case where the cysteine unit is present terminally in the compound of the formula (I), one of the dashed lines corresponds to hydrogen or —OH, depending on whether the cysteine unit is covalently bound with further structural units via the carboxylic acid function or via the amine function. For example, the cysteine unit in the following compound of the formula (IV) is positioned terminally, covalently bound with further structural units the carboxylic acid function (with formation of an amide) and the amine function of the cysteine unit is present as NH₂.

In the production of a multicomponent adhesive according to the invention and the production of an adhesive hydrogel according to the invention (see also examples 2 and 3) the presence of a thiol group (SH group) in the compound of the formula (I) enables the Michael addition at a pH at physiological level (pH 6 to 7.5); at the same time, at such a pH, autoxidation of the dihydroxy group is prevented.

Multicomponent adhesives according to the invention in which the compound of the formula (I) contains specific amino acid units or combinations of certain amino acid units, are characterized by especially good biocompatibility and/or are biodegradable. In addition, in the cases in which the compound of the formula (I) contains specific amino acid units or combinations of certain amino acid units, the adhesive hydrogel can be prepared from the multicomponent adhesive according to the invention by crosslinking without addition of an oxidizing agent or enzyme. Preferably a multicomponent adhesive according to the invention, wherein the linker L in the compound of the formula (I) contains one, two or more structural unit, is selected from the group consisting of glycine, alanine, valine, leucine, isoleucine, methionine, phenylalanine, tryptophan, proline, serine, threonine, cysteine, tyrosine, asparagine, glutamine, aspartic acid, glutamic acid, lysine, arginine and histidine, preferably selected from the group consisting of cysteine, alanine, lysine, proline, serine, tyrosine and threonine.

Especially preferably, a multicomponent adhesive according to the invention, in which the linker L in the compound of the formula (I) contains one, two or more structural units is selected from the group of amino acid sequences consisting of alanine-lysine, lysine-proline, proline-serine, serine-tyrosine, tyrosine-proline, proline-proline, proline-threonine, alanine-lysine-proline, lysine-proline-serine, proline-serine-tyrosine, serine-tyrosine-proline, tyrosine-proline-proline, proline-proline-threonine, alanine-lysine-proline-serine (SEQ ID NO: 1), lysine-proline-serine-tyrosine (SEQ ID NO: 2), proline-serine-tyrosine-proline (SEQ ID NO: 3), serine-tyrosine-proline-proline (SEQ ID NO: 4), tyrosine-proline-proline-threonine (SEQ ID NO: 5), alanine-lysine-proline-serine-tyrosine (SEQ ID NO: 6), lysine-proline-serine-tyrosine-proline (SEQ ID NO: 7), proline-serine-tyrosine-proline-proline (SEQ ID NO: 8), serine-tyrosine-proline-proline-threonine (SEQ ID NO: 9), alanine-lysine-proline-serine-tyrosine-proline (SEQ ID NO: 10), lysine-proline-serine-tyrosine-proline-proline (SEQ ID NO: 11), proline-serine-tyrosine-proline-proline-threonine (SEQ ID NO: 12), alanine-lysine-proline-serine-tyrosine-proline-proline (SEQ ID NO: 13), lysine-proline-serine-tyrosine-proline-proline-threonine (SEQ ID NO: 14) and alanine-lysine-proline-serine-tyrosine-proline-proline-threonine (SEQ ID NO: 15).

The “SEQ ID NO” relate to the appended sequence protocol, which is a constituent of the present description.

A multicomponent adhesive according to the invention in which the compound of the formula (I) has the structure according to formula (IV) (SEQ ID NO: 16) is especially preferred:

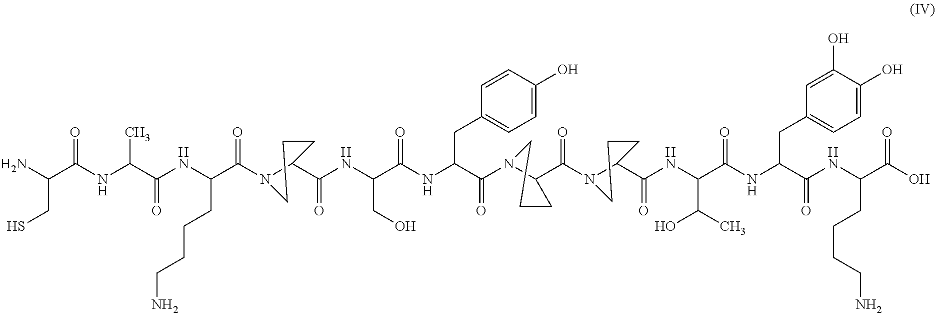 [SMILES: CC(NC(=O)C(N)CS)C(=O)NC(CCCCN)C(=O)N1CCCC1C(=O)NC(CO)C(=O)NC(Cc1ccc(O)cc1)C(=O)N1CCCC1C(=O)N1CCCC1C(=O)NC(C(=O)NC(Cc1ccc(O)c(O)c1)C(=O)NC(CCCCN)C(=O)O)C(C)O]

The adhesive hydrogel according to the invention resulting from a multicomponent adhesive according to the invention containing the compound of the formula (IV) displays very good adhesion to biological tissue of allogenic, heterogenic or synthetic origin and/or to medical implants, good biocompatibility and good biodegradability.

The compound of the formula (IV) is a preferred example of a compound of the formula (I).

A multicomponent adhesive according to the invention, in which, if X in the compound of the formula (I) means an alpha,beta-unsaturated carbonyl group, X is selected from the group consisting of alpha,beta-unsaturated aldehydes, alpha,beta-unsaturated ketones, alpha,beta-unsaturated esters, preferably acrylate esters, and alpha,beta-unsaturated carboxylic acid amides, preferably maleimide, is preferred.

### Constituent b), Compound Containing the Structural Unit B-L′-Y (II):

In our own studies, it has also been found that the linker L′ in the structural unit B-L′-Y (II) has an influence on the properties (e.g. biocompatibility, biodegradability, good adhesion to biological tissue of allogenic, heterogenic or synthetic origin and/or to medical implants) of the multicomponent adhesive or the adhesive hydrogel resulting therefrom. A multicomponent adhesive according to the invention in which the linker L′ in the structural unit B-L′-Y (II) is an organic molecule unit, the chain members whereof consist of (a) exclusively carbon atoms or (b) carbon atoms and hetero atoms, is preferred. Such a multicomponent adhesive according to the invention or the adhesive hydrogel according to the invention resulting therefrom displays good biocompatibility, biodegradability and/or little or no toxicity.

In the context of the present text, a chain member of the linker L′ is understood to mean a single atom (chain atom or chain member), which in combination with further atoms consecutively linked by covalent bonds (further chain atoms or chain members) links B by the shortest route with the functional group Y. Only the atoms which lie on the shortest route between B and X are counted as chain members; atoms in side chains are not considered. For example the number of the chain members of a linker L′ in the following compound of the formula (V) is 7, when n corresponds to 1 and m to 2 (this is the number of the atoms between the group Y and the linkage point B).

B is a linkage point which links the individual groups L′-Y to one another. Usually B is a carbon atom, a nitrogen atom or an oxygen atom. For example in the following compound of the formula (V), B corresponds to the quaternary carbon atom, to which four groups L′-Y are bonded.

A multicomponent adhesive according to the invention, in which, if Y in the structural unit B-L′-Y (II) means an alpha,beta-unsaturated carbonyl group, Y is selected from the group of alpha,beta-unsaturated aldehydes, alpha,beta-unsaturated ketones, alpha,beta-unsaturated esters, preferably acrylate esters, and alpha,beta-unsaturated carboxylic acid amides, preferably maleimide, is preferred

A multicomponent adhesive according to the invention is especially preferable, when the linker L′ in the structural unit B-L′-Y (II) is or comprises a hydrocarbon, a polyether or a peptide. The adhesive hydrogels resulting from such multicomponent adhesives according to the invention always display good biocompatibility.

A multicomponent adhesive according to the invention is also especially preferred, if the structural unit (II) has the following structure (IIa):

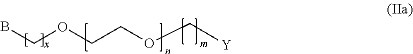

wherein n is a whole number greater than or equal to 1, x is 0, 1, 2, 3, 4, 5, 6, 7, or 8 and m is 0, 1, 2, 3, 4, 5, 6, 7, or 8. The adhesive hydrogels resulting from such multicomponent adhesives according to the invention display very good water absorption capacity and very good biocompatibility.

The structural unit (IIa) is a preferred example of a the structural unit B-L′-Y (II).

Especially preferred are multicomponent adhesives according to the invention, containing in or as constituent (b) one, two, three, four or more compounds selected from compounds of the formula (V)

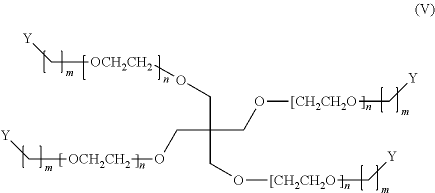

wherein
n in each of the four side chains independently of the meaning of n in the respective other side chains is a whole number greater than or equal to 1 and
m in each of the four side chains independently of the meaning of m in the respective other side chains is 0, 1, 2, 3, 4, 5, 6, 7, or 8
and
Y in each of the four side chains independently of the meaning of Y in the respective other side chains is selected from the group consisting of SH, NH₂ and alpha,beta-unsaturated carbonyl groups and can be reacted with the group X with formation of at least one covalent bond.

The compound of the formula (V) is a preferred example of a compound containing the structural unit B-L′-Y (II) or a compound containing the structural unit (IIa).

Quite especially preferred is a multicomponent adhesive according to the invention as just defined, wherein in the compound of the formula (V)

m in each of the four side chains is identical
and/or
Y in each of the four side chains is identical.

Since the compound of the formula (V) in the especially preferred configurations exhibits high symmetry and all side arms (as example for structural units (II)) are identical, this compound can be particularly easily produced. Thereby for example the costs of the compound with the formula (V) and thus also the costs of a multicomponent adhesive according to the invention can be reduced. Through the simple synthetic access to compounds of the formula (V) in few reaction steps, the compound of the formula (V) can also be obtained in high purity. Impurities and side products, which would inevitably arise in reactions with many synthesis steps and cannot be removed, are avoided. As a result, a multicomponent adhesive according to the invention can be obtained in very good purity and quality.

Particularly preferred is a multicomponent adhesive according to the invention as just defined, containing in or as constituent (b) a compound of the formula (VI)

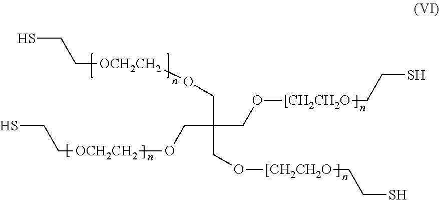

wherein
n in each of the four side chains independently of the meaning of n in the respective other side chains is a whole number greater than or equal to 1.

The compound of the formula (VI) is an especially preferred example of a compound of the formula (V) and an especially preferred configuration of a compound containing the structural unit B-L′-Y (II) or the structural unit (IIa).

The SH groups of the compound of the formula (VI) (and similar compounds) react very selectively, with high yields and under mild conditions with alpha,beta-unsaturated carbonyl groups. Thus the corresponding multicomponent adhesives according to the invention and the adhesive hydrogels formed therefrom exhibit very high quality.

Especially preferable is a multicomponent adhesive according to the invention, wherein one, two, three, four or all compounds, which independently of one another each comprise two, three, four, five, six, seven, eight or more structural units (II), (a) have a weight average of the molecular mass (M_{w}) in the range from 500 to 40000 g/mol, preferably in the range from 500 to 25000 g/mol, especially preferably in the range from 1000 to 15000 g/mol, particularly preferably in the range from 2000 to 8000 g/mol and/or (b) have a polydispersity of less than 4, preferably less than 3, especially preferably less than 2, particularly preferably less than 1.5.

Constituent (ii), Compound Containing the Structural Unit B′-L″-Z′ (III):

In our own studies, it has also been found that the linker L″ in structural unit B′-L″-Z′ (III) has an influence on the properties (e.g. biocompatibility, biodegradability, good adhesion to biological tissue of allogenic, heterogenic or synthetic origin and/or to medical implants) of the multicomponent adhesive or the adhesive hydrogel resulting therefrom. A multicomponent adhesive according to the invention is preferred if the linker L″ in the structural unit B′-L″-Z′ (III) is an organic molecule unit, the chain members whereof consist of (a) exclusively carbon atoms or (b) carbon atoms and hetero atoms. As a result, the multicomponent adhesive according to the invention or the adhesive hydrogel according to the invention resulting therefrom exhibits good biocompatibility, biodegradability and/or little or no toxicity.

In the context of the present text, a chain member of the linker L″ is understood to mean a single atom (chain atom or chain member), which in combination with further atoms consecutively linked by covalent bonds (further chain atoms or chain members) links B by the shortest route with the functional group Z. Only the atoms which lie on the shortest route between the B′ and Z′ are counted as chain members; atoms in side chains are not considered. For example, the number of the chain members of a linker L″ in the following compound of the formula (VII) is 5, if o corresponds to 1.

B′ is a linkage point, which binds individual groups L″-Z′ to one another. Usually B is a carbon atom, a nitrogen atom or an oxygen atom For example, in the following compound of the formula (VII) B′ corresponds to the oxygen to which two structural units (III) are bound.

A multicomponent adhesive according to the invention, in which, if Z′ in structural unit B′-L″-Z′ (III) is an alpha,beta-unsaturated carbonyl group, Z′ is selected from the group of alpha,beta-unsaturated aldehydes, alpha,beta-unsaturated ketones, alpha,beta-unsaturated esters, preferably acrylate esters, and alpha,beta-unsaturated carboxylic acid amides, preferably maleimide, is preferred.

A multicomponent adhesive according to the invention is especially preferred if the linker L″ in the structural unit B′-L″-Z′ (III) is or comprises a hydrocarbon, a polyether or a peptide. The adhesive hydrogels resulting from these multicomponent additives according to the invention exhibit very good biocompatibility.

A multicomponent adhesive according to the invention is also especially preferred, when the structural unit (III) has the following structure (IIIa):

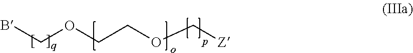

wherein o is a whole number greater than or equal to 1, q is 0, 1, 2, 3, 4, 5, 6, 7, or 8 and p is 0, 1, 2, 3, 4, 5, 6, 7, or 8. The adhesive hydrogels resulting from these multicomponent additives according to the invention display very good water absorption capacity and very good biocompatibility.

The structural unit (IIIa) is a preferred example of a the structural unit B′-L″-Z′ (III).

Especially preferred is a multicomponent adhesive according to the invention, containing in or as compound comprising the structural unit (III) one, two, three, four or more compounds selected from compounds according to formula (VII)

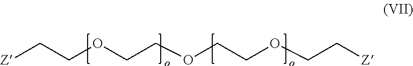

wherein o in each of the two side chains independently of the meaning of o in the respective other side chains is a whole number greater than or equal to 1 and

Z′ of each of the two side chains independently of the meaning of Z′ in the respective other side chains is selected from the group consisting of SH, NH₂ and alpha,beta-unsaturated carbonyl groups and can be reacted with a group Y with formation of at least one covalent bond.

The compound of the formula (VII) is a preferred example of a compound containing the structural unit B′-L″-Z′ (III) or a compound containing the structural unit (IIIa).

Since the compound of the formula (VII) exhibits high symmetry and both side arms (structural units (III)) are identical, this compound can be particularly easily produced. As a result, for example the costs of the compound with the formula (VII) and thus also the costs of a multicomponent adhesive according to the invention can be reduced. Through the simple synthetic access to compounds of the formula (VII) in few reaction steps, the compound of the formula (VII) can also be obtained in high purity. Impurities and side products, which would inevitably arise in reactions with many synthesis steps and cannot be removed, are avoided. As a result, a multicomponent adhesive according to the invention can be obtained in very good purity and quality.

Quite especially preferred is a multicomponent adhesive according to the invention, containing in or as constituent (ii) a compound of the formula (VIII)

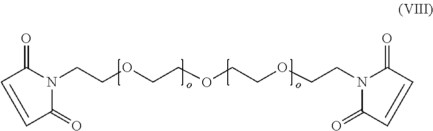

wherein o is a whole number greater than or equal to 1.

The compound of the formula (VIII) is an especially preferred example of a compound of the formula (VII) and an especially preferred configuration of a compound containing the structural unit B′-L″-Z′ (III) or the structural unit (IIIa).

The alpha,beta-unsaturated carbonyl groups of the compound with the formula (VIII) are maleimide groups. These maleimide groups react very selectively, with high yields and under mild conditions with Michael donors such as SH and NH₂, in particular with SH. Accordingly, the resulting multicomponent adhesives according to the invention and the adhesive hydrogels according to the invention arising therefrom exhibit very high quality.

Especially preferable is a multicomponent adhesive according to the invention, wherein one, two, three, four or all compounds, which independently of one another each comprise two, three, four, five, six, seven, eight or more structural units (III), (a) have a weight average of the molecular mass (M_{w}) in the range from 500 to 40000 g/mol, preferably in the range from 500 to 25000 g/mol, especially preferably in the range from 1000 to 15000 g/mol, particularly preferably in the range from 2000 to 8000 g/mol and/or (b) have a polydispersity of less than 4, preferably less than 3, especially preferably less than 2, particularly preferably less than 1.5.

The weight average of the molecular mass (M_{w}) is determined by gel permeation chromatography with polystyrene (PS) as standard. The polydispersity corresponds to the quotient of the mass average of the molecular masses (M_{w}) and the number average of the molecular mass (Mₙ).

The individual (in particular the preferred) configurations (as described above) of the constituents a), b), c), d) and (ii) discussed in detail above of a multicomponent adhesive according to the invention can be combined with one another.

Especially preferred is a multicomponent adhesive according to the invention for producing an adhesive hydrogel, comprising
a) a compound of the formula (IV) (SEQ ID NO: 16) (as an example of a compound of the formula (I))

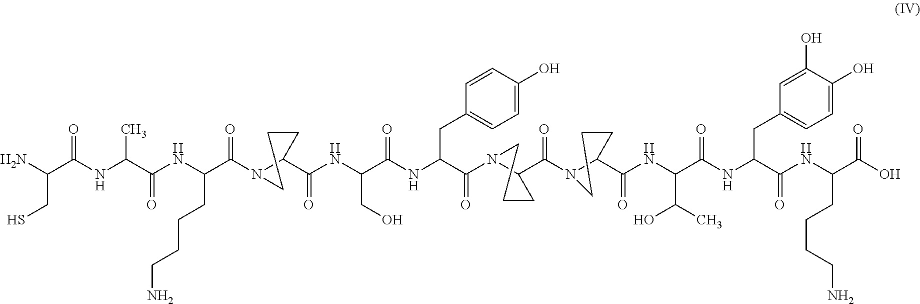 [SMILES: CC(NC(=O)C(N)CS)C(=O)NC(CCCCN)C(=O)N1CCCC1C(=O)NC(CO)C(=O)NC(Cc1ccc(O)cc1)C(=O)N1CCCC1C(=O)N1CCCC1C(=O)NC(C(=O)NC(Cc1ccc(O)c(O)c1)C(=O)NC(CCCCN)C(=O)O)C(C)O]

and
b) a compound of the formula (VI) (as an example of a compound of the formula (II))

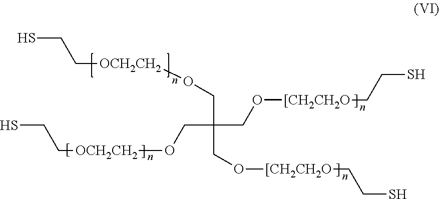

wherein
n in each of the four side chains independently of the meaning of n in the respective other side chains is a whole number greater than or equal to 1
   and
   a compound of the formula (VIII) (as an example of a compound of the formula (III))

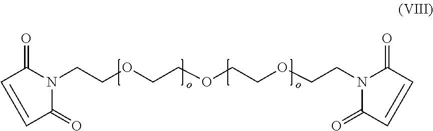

wherein o is a whole number greater than or equal to 1
   and
c) a buffer consisting of a buffer acid and a corresponding buffer base, where the pK_{B} of the buffer base is not less than 2, preferably is not less than 4
   and
d) one or more bases with a pK_{B}<1,
   wherein one, two, three or all components of the multicomponent system have a pH in the range from 6 to 8.

A multicomponent adhesive according to the invention which is biocompatible and/or biodegradable is preferred.

In this context, the term “biocompatible” is understood to mean the ability of the multicomponent adhesive to have no or only a slight adverse effect on living tissue in the vicinity of the multicomponent adhesive. “Biocompatible” are on the one hand multicomponent adhesives, in the biological assessment whereof according to DIN EN ISO 10993-1:2010-04, no chronic toxicity, carcinogenicity, toxicokinetics, immunotoxicity, reproductive/developmental toxicity and other organ-specific toxicity is detected, and on the other hand other biocompatible multicomponent adhesives, in particular those multicomponent adhesives with which adverse effects are at least compensated by positive effects.

In this context, the term “biodegradable” is understood to mean the ability of the multicomponent adhesive to be biologically degraded. “Biodegradable” are on the one hand “enzymatically degradable” multicomponent adhesives, that is destroyable by enzymes, and on the other hand multicomponent adhesives biologically degradable in another way, in particular multicomponent adhesives hydrolytically degradable by biological routes.

A preferred biodegradable multicomponent adhesive according to the invention has an inherent degradability (preferably according to test methods of the Organization for Economic Cooperation and Development “OECD 302”).

A multicomponent adhesive according to the invention for use in a method for surgical or therapeutic treatment of the human or animal body is preferred.

Likewise preferred is a multicomponent adhesive according to the invention, for gluing of (a) medical and/or (b) metallic implants, preferably medical, metallic implants, which preferably have a titanium content of more than 89 wt. %, based on the total mass of the implant, to human or animal tissue, wherein the tissue is preferably selected from the group consisting of bone, mucosa, gingiva, skin, cartilage, tooth, periodontium, connective tissue, muscle tissue, nerve tissue and epithelial tissue.

Likewise preferred is a multicomponent adhesive according to the invention for gluing together two parts of human or animal tissue, wherein (i) each of the two tissues independently of the respective other tissue is preferably selected from the group consisting of bone, mucosa, gingiva, skin, cartilage, dental substance, periodontium, connective tissue, muscle tissue, nerve tissue and epithelial tissue, and/or (ii) the tissue is allogenically, heterogenically, or at least one tissue synthetically or biologically producible. A purely artificially produced tissue without living additives (e.g. proteins) or starting materials (e.g. cells) is a synthetic tissue; a production method for tissue, in which living cells or e.g. bacteria are used is a biological production method, which leads to biologically producible tissue.

Likewise preferred is a multicomponent adhesive according to the invention for gluing together (a) an implant with gingiva, (b) an implant with a biomaterial (allogenic, heterogenic or synthetic), (c) an implant with bone, (d) a biomaterial (allogenic, heterogenic or synthetic) with gingiva, (e) a biomaterial (allogenic, heterogenic or synthetic) with bone, (f) a biomaterial (allogenic, heterogenic or synthetic) with dental substance, (g) a biomaterial (allogenic, heterogenic or synthetic) with periodontium, (h) bone with gingiva, (i) bone with periodontium, (j) dental substance with gingiva, (k) dental substance with periodontium, (l) periodontium with gingiva, (m) an implant with a chemical or biological treatment material, (n) a biomaterial (allogenic, heterogenic or synthetic) with a chemical or biological treatment material, (o) bone with a chemical or biological treatment material, (p) dental substance with a chemical or biological treatment material or (q) periodontium with a chemical or biological treatment material.

A multicomponent adhesive according to the invention, comprising the compound of the formula (I) in a quantity from 1 to 15 wt. %, preferably in a quantity from 3 to 10 wt. %, preferably in a quantity from 4 to 7 wt. %, based on the total mass of the compound of the formula (I), of the compound(s) each containing one, two, three, four, five, six, seven, eight or more structural units (II), and of the compound(s) each containing one, two, three, four, five, six, seven, eight or more structural units (III), is preferred.

Especially preferred is a multicomponent adhesive according to the invention, comprising
the compound of the formula (I), wherein the total quantity of the compound of the formula (I) lies in the range from 1 to 15 wt. %, preferably in the range from 3 to 10 wt. %, preferably in the range from 4 to 7 wt. %, based on the total mass of the compound of the formula (I), of the compound(s) each containing one, two, three, four, five, six, seven, eight or more structural units (II), and of the compound(s) each containing one, two, three, four, five, six, seven, eight or more structural units (III),
one, two, three, four or more compounds, each containing one, two, three, four, five, six, seven, eight or more structural units (II), wherein the total quantity of the compounds containing one, two, three, four or more structural units (II) lies in the range from 55 to 70 wt. %, preferably in the range from 60 to 67 wt. %, preferably in the range from 63 to 66 wt. %, based on the total mass of the compound of the formula (I), of the compound(s) each containing one, two, three, four, five, six, seven, eight or more structural units (II), and of the compound(s) each containing one, two, three, four, five, six, seven, eight or more structural units (III), and
additionally comprises one, two, three, four or more compounds, which independently of one another each contain two, three, four, five, six, seven, eight or more structural units (III), wherein the total quantity of the compound containing respectively two, three, four, five, six, seven, eight or more structural units (III) lies in the range from 20 to 44 wt. %, preferably in a quantity from 27 to 35 wt. %, preferably in a quantity from 28 to 31 wt. %, based on the total mass of the compound of the formula (I), of the compound(s) each containing one, two, three, four, five, six, seven, eight or more structural units (II), and of the compound(s) each containing one, two, three, four, five, six, seven, eight or more structural units (III).

The total mass of the compound of the formula (I), of the compound(s) each containing one, two, three, four, five, six, seven, eight or more structural units (II), and of the compound(s) each containing one, two, three, four, five, six, seven, eight or more structural units (III), is the (total) sum of the masses of these compounds.

The invention further relates to the use of a multicomponent adhesive according to the invention for non-therapeutic gluing together of two surfaces.

The invention also relates to a method for producing an adhesive hydrogel comprising the following steps:
Provision or production of a multicomponent adhesive according to the invention (as described above; preferably as designated above as preferred; as regards preferred constituents, that stated further above applies),
Allowing the components of the multicomponent adhesive to react, so that the adhesive hydrogel is formed.

For the method according to the invention described above, it preferably applies that the adhesive hydrogel formation takes place by crosslinking of the aforesaid groups X, Y, Z and Z′ of the constituents of the multicomponent adhesive.

For the methods according to the invention described above, it especially preferably applies that the adhesive hydrogel formed is then treated with an oxidizing agent, preferably with NaIO₄, H₂O₂ or with an oxidoreductase, preferably one with the EC Number 1.10 (in particular laccase, catechol oxidase, tyrosinase, phenol oxidase), preferably with NaIO₄ or H₂O₂, so that an additional crosslinking takes place.

In some configurations it is especially preferable already to add the appropriate oxidizing agent during the mixing or shortly after the mixing of all components of the multicomponent adhesive according to the invention.

The adhesive hydrogel treated with an oxidizing agent exhibits especially good properties. In particular, good adhesion to biological tissue of allogenic, heterogenic or synthetic origin and to medical implants and good barrier action against bacteria is achieved.

The invention further relates to a kit comprising a multicomponent adhesive according to the invention (as described above; preferably as designated above as preferred; as regards preferred constituents, that stated further above applies) and one or more further components.

A kit according to the invention, in which the one or more further components is or are selected from the group consisting of syringes, cannulas, implants, stents, catheters, applicator, mixing device (e.g. static or dynamic mixer) and oxidizing agents for crosslinking an adhesive hydrogel produced from the multicomponent adhesive, is preferred.

The invention also relates to a method for therapeutic or non-therapeutic gluing together of two or more surfaces, comprising the following steps:
Provision or production of a multicomponent adhesive according to the invention (as described above; preferably as designated above as preferred; as regards preferred constituents, that stated further above applies),
Provision or production of two or more surfaces,
Application of a mixture of the components of the produced or provided multicomponent adhesive onto one, two or more of the provided or produced surfaces,
Production of a bond comprising two or more of the provided or produced surfaces and between these surfaces and a mixture of the components of the produced or provided multicomponent adhesive binding these.
Allowing the mixture of the components of the multicomponent adhesive to react in said bond so that an adhesive hydrogel is formed and the surfaces of the bond are glued together.

A method according to the invention in which at least one of the provided or produced surfaces is the surface of an element with a titanium content of more than 89 wt. %, based on the total mass of the element, is preferred.

The invention also relates to an adhesive hydrogel producible from a multicomponent adhesive according to the invention (as described above; preferably as designated above as preferred; as regards preferred constituents, that stated further above applies), preferably produced by a method according to the invention for production of an adhesive hydrogel.

The invention likewise relates to an article comprising two surfaces glued together, between which an adhesive hydrogel producible from a multicomponent adhesive according to the invention (as described above; preferably as designated above as preferred; as regards preferred constituents, that stated further above applies) is arranged and the adhesive bond effected.

The invention likewise relates to an article comprising a surface on which an adhesive hydrogel producible from a multicomponent adhesive according to the invention (as described above; preferably as designated above as preferred; as regards preferred constituents, that stated further above applies) is positioned in adhesive bonding.

The invention likewise relates to a compound of the formula (IV) (SEQ ID NO: 16)

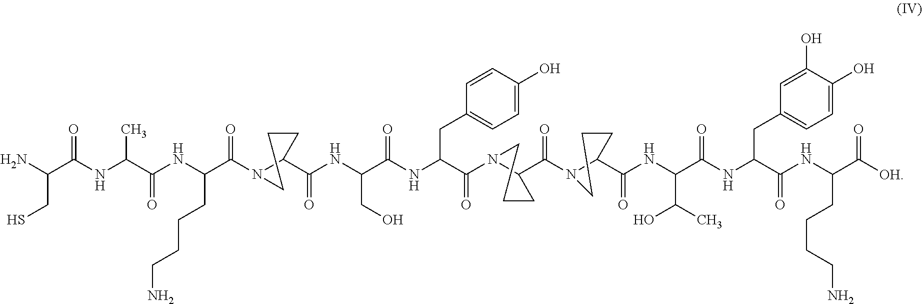 [SMILES: CC(NC(=O)C(N)CS)C(=O)NC(CCCCN)C(=O)N1CCCC1C(=O)NC(CO)C(=O)NC(Cc1ccc(O)cc1)C(=O)N1CCCC1C(=O)N1CCCC1C(=O)NC(C(=O)NC(Cc1ccc(O)c(O)c1)C(=O)NC(CCCCN)C(=O)O)C(C)O]

The following examples illustrate the invention; unless otherwise stated, all data relate to weight.

### EXAMPLES

### Example 1

### Production of a Compound According to the Invention of the Formula (IV) (Decapeptide) (SEQ ID NO: 16)

For the synthesis, a TCP-polystyrene resin preloaded with Fmoc-amino acids was used on a 433A Solid Phase Peptide Synthesizer (SPPS) from Applied Biosystems (Farmington, USA) was used. The syntheses were performed on the 0.1 mmol scale. Substitution level for lysine=0.54 mmol/g. The FastMoc protocol was followed. This protocol utilizes the orthogonal protective group strategy: The -amine is equipped with a temporary protective group. This function is fulfilled by the fluorenylmethoxycarbonyl (Fmoc). At the same time, amino acids which have reactive groups on their side chains are protected with permanent protective groups against the synthesis chemistry. The Fmoc-protected N-terminus of the L-amino acids used was deprotected with 20% piperidine in NMP after each coupling cycle and, when the resins were used already loaded with amino acid, additionally before the first coupling. The base-stable side chain protective groups were removed after the solid phase synthesis. The coupling of an amino acid onto the growing substance on the resin is effected by a nucleophilic attack of the N-terminus on the carbonyl carbon of the amino acid to be coupled. This carbon is subjected beforehand to the standardized activation with HBTU or with HATU in NMP. The removal of the resin and the permanent protective groups was effected in one step by addition of 400 μL of a trifluoroacetic acid/triisopropylsilane/water mixture in the ratio (18:1:1). After 2 hours, the removed peptide was transferred through a syringe filter (0.45 μm) into ice-cold tert-butyl methyl ether (MTBE). The peptide precipitated therein was further precipitated for a further two hours at −20° C. Next, the solution was centrifuged. The residue was dissolved in water, transferred into a flask and lyophilized. The compound of the formula (IV) (below also: decapeptide) is obtained in high purity.

### Example 2

### Production of a Multicomponent Adhesive According to the Invention and Production of an Adhesive Hydrogel

In one 500 μl microreaction vessel each, 15 mg (ca. 7.5 μmol) of 4-Arm-PEG-SH (compound VI; M_{w}=2000 g/mol; (4 arm PEG SH (pentaerythritol))) are dissolved in 150 μl 0.1M carbonate buffer (adjusted with 1M HCl to pH 7.5) and 7 mg (ca. 3.5 μmol) of Mal-PEG-Mal (compound VIII; M_{w}=1930 g/mol; alpha,omega-bis-maleimido poly(ethylene glycol)) in 200 μl 0.1M carbonate buffer (adjusted with 1M HCl to pH 7.5). To the solution of Mal-PEG-Mal is added a further solution consisting of 1 mg (0.7 μmol) of decapeptide (compound (IV)) dissolved in 50 μl 0.1M carbonate buffer (the pH of the finished solution is adjusted with ca. 1 μl NaOH solution to pH 7.5) and stirred well with a vortex mixer. The two solutions produced constitute a multicomponent adhesive according to the invention. After 10 minutes stirring time, the two solutions consisting of a) the Mal-PEG-Mal and the decapeptide and b) 4-Arm-PEG-SH are applied as simultaneously as possible onto a substrate by means of two pipettes. An adhesive hydrogel is formed, which reaches its final strength after ca. 5 minutes.

### Example 3

### Production of a Multicomponent Adhesive According to the Invention and Production of an Adhesive Hydrogel with Crosslinking

In one 500μl microreaction vessel each, 15 mg (ca. 7.5 μmol) of 4-Arm-PEG-SH (compound VI; M_{w}=2000 g/mol; (4 arm PEG SH (pentaerythritol))) are dissolved in 150 μl 0.1M carbonate buffer (adjusted with 1M HCl to pH 7.5) and 7 mg (ca. 3.5 μmol) of Mal-PEG-Mal (compound VIII; M_{w}=1930 g/mol; alpha,omega-bis-maleimido poly(ethylene glycol)) in 200 μl 0.1M carbonate buffer (adjusted with 1M HCl to pH 7.5). To the solution of Mal-PEG-Mal is added a further solution consisting of 1 mg (0.7 μmol) of decapeptide (compound (IV)) dissolved in 50 μl 0.1M carbonate buffer (the pH of the finished solution is adjusted with ca. 1 μl NaOH solution to pH 7.5) and stirred well with a vortex mixer. The two solutions produced constitute a multicomponent adhesive according to the invention. After 10 minutes stirring time, the two solutions consisting of a) the Mal-PEG-Mal and the decapeptide and b) 4-Arm-PEG-SH are applied as simultaneously as possible onto a substrate by means of two pipettes. Directly after the application, 10 μl of a saturated sodium periodate solution are pipetted into the adhesive hydrogel forming. An adhesive hydrogel is formed, which reaches its final strength after ca. 5 minutes.

### Example 4

### Adhesive Strength of an Adhesive Hydrogel According to the Invention

A multicomponent adhesive produced according to example 2 is applied onto a freshly removed piece of pig gingiva as substrate and, immediately after the application, covered with a titanium substrate, so that an adhesive bond between titanium substrate and pig gingiva is formed. An adhesive gel is formed, which reaches its final strength after ca. 5 minutes.

In a tensile test, the tensile strength of the adhesive hydrogel formed between the titanium substrate and the pig gingiva is tested.

The adhesive hydrogel formed was found to have sufficient tensile strength for the intended purposes.

### Example 5

### Tensile Strength of an Adhesive Hydrogel According to the Invention

A multicomponent adhesive produced according to example 2 is applied onto a freshly removed piece of pig gingiva as substrate. Directly after the application, 10 μl of a saturated sodium periodate solution are pipetted onto the adhesive hydrogel forming. Directly after the application of the sodium periodate solution, the multicomponent adhesive (or the adhesive hydrogel forming) is covered with a titanium substrate, so that an adhesive bond between titanium substrate and pig gingiva is formed. An adhesive gel is formed, which reaches its final strength after ca. 5 minutes.

In a tensile test, the tensile strength of the adhesive hydrogel formed between the titanium substrate and the pig gingiva is tested.

The adhesive hydrogel formed was found to have sufficient tensile strength for the intended purposes.

### Comparative Example 1

### Production of a Hydrogel from a Two-Component System

In one 500 μl microreaction vessel each, 7 mg (ca. 3.5 μmol) of Mal-PEG-Mal (compound VIII; M_{w}=2000 g/mol) and 15 mg (ca. 7.5 μmol) of 4-Arm-PEG-SH (compound VI; M_{w}=2000 g/mol) are each dissolved in 200 μl 0.1M carbonate buffer (adjusted with 1M HCl to pH 7.5). The two solutions consisting of a) the Mal-PEG-Mal and b) the 4-Arm-PEG-SH are applied as simultaneously as possible onto a substrate by means of two pipettes. After ca. 5 minutes, the adhesive hydrogel formed has reached its final strength.

### Comparative Example 2

### Tensile Strength of a Hydrogels

A two-component system produced according to comparative example 1 is applied onto a freshly removed piece of pig gingiva as substrate and directly after the application covered with a titanium substrate, so that an adhesive bond between titanium substrate and pig gingiva is formed. After ca. 5 minutes, the hydrogel formed has reached its final strength.

In a tensile test, the tensile strength of the hydrogel formed between the titanium substrate and the pig gingiva is tested.

The adhesive hydrogel formed does not have sufficient tensile strength for the intended purposes.

1. A multicomponent adhesive for producing an adhesive hydrogel, comprising
a) a first compound of the formula (I) 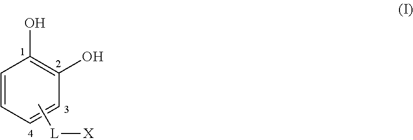
wherein
X is selected from the group consisting of SH, NH₂ and alpha,beta-unsaturated carbonyl groups, and
L is a linker which links the 1,2-dihydroxyphenyl unit with X, wherein the linker L is linked with the 1,2-dihydroxyphenyl unit in the 3 or 4 position;
and
b) at least one second compound containing at least one structural unit (II)
B-L′-Y (II)
wherein in each of the structural units (II) independently of the meaning in other structural units (II)
B is a linkage point,
L′ is a linker, and
Y is selected from the group consisting of SH, NH₂ and alpha,beta-unsaturated carbonyl groups and can be reacted with the group X with formation of at least one covalent bond;
and
c) at least one of
(i) the second compound additionally comprising a residue Z, which independently of every other residue Z of another second compound is selected from the group consisting of SH, NH₂ and alpha,beta-unsaturated carbonyl groups and can be reacted with a group Y with formation of at least one covalent bond,
and
(ii) at least one third compound, which independently of another third compound comprises at least two structural units (III)
B′-L″-Z′ (III)
wherein in each of the structural units (III) independently of the meaning in other structural units (III)
B′ is a linkage point,
L″ is a linker, and
Z′ is selected from the group consisting of SH, NH₂ and alpha,beta-unsaturated carbonyl groups and can be reacted with a group Y with formation of at least one covalent bond.

2. The multicomponent adhesive as claimed in claim 1, further comprising at least one of
d) a buffer consisting of a buffer acid and a corresponding buffer base, where the pK_{B} of the buffer base is not less than 2,
and
e) one or more bases with a pK_{B}<1.

3. The multicomponent adhesive as claimed in claim 1, wherein the first compound of the formula (I) contains at least one cysteine unit.

4. The multicomponent adhesive as claimed in claim 3, wherein the first compound of the formula (I) contains at least one L-cysteine unit (formula X).

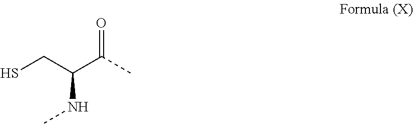

5. The multicomponent adhesive as claimed in claim 1, wherein at least one of
(i) the linker L of the first compound of the formula (I) is an organic molecule unit, the chain members whereof consisting of at least one of (a) exclusively carbon atoms and (b) carbon atoms and hetero atoms,
(ii) the linker L′ in the structural unit B-L′-Y (II) is an organic molecule unit, the chain members whereof consisting of at least one of (a) exclusively carbon atoms and (b) carbon atoms and hetero atoms,
and
(iii) the linker L″ in the structural unit B′-L″-Z′ (III) is an organic molecule unit, the chain members whereof consisting of at least one of (a) exclusively carbon atoms and (b) carbon atoms and hetero atoms.

6. The multicomponent adhesive as claimed in claim 1, wherein the linker L in the first compound of the formula (I) contains at least one structural unit selected from the group consisting of glycine, alanine, valine, leucine, isoleucine, methionine, phenylalanine, tryptophan, proline, serine, threonine, cysteine, tyrosine, asparagine, glutamine, aspartic acid, glutamic acid, lysine, arginine and histidine.

7. The multicomponent adhesive as claimed in claim 6, wherein the linker L in the first compound of the formula (I) contains at least one structural unit selected from the group consisting of cysteine, alanine, lysine, proline, serine, tyrosine and threonine.

8. The multicomponent adhesive as claimed in claim 1, wherein the linker L in the first compound of the formula (I) contains at least one structural unit selected from the group of amino acid sequences consisting of alanine-lysine, lysine-proline, proline-serine, serine-tyrosine, tyrosine-proline, proline-proline, proline-threonine, alanine-lysine-proline, lysine-proline-serine, proline-serine-tyrosine, serine-tyrosine-proline, tyrosine-proline-proline, proline-proline-threonine, alanine-lysine-proline-serine (SEQ ID NO: 1), lysine-proline-serine-tyrosine (SEQ ID NO: 2), proline-serine-tyrosine-proline (SEQ ID NO: 3), serine-tyrosine-proline-proline (SEQ ID NO: 4), tyrosine-proline-proline-threonine (SEQ ID NO: 5), alanine-lysine-proline-serine-tyrosine (SEQ ID NO: 6), lysine-proline-serine-tyrosine-proline (SEQ ID NO: 7), proline-serine-tyrosine-proline-proline (SEQ ID NO: 8), serine-tyrosine-proline-proline-threonine (SEQ ID NO: 9), alanine-lysine-proline-serine-tyrosine-proline (SEQ ID NO: 10), lysine-proline-serine-tyrosine-proline-proline (SEQ ID NO: 11), proline-serine-tyrosine-proline-proline-threonine (SEQ ID NO: 12), alanine-lysine-proline-serine-tyrosine-proline-proline (SEQ ID NO: 13), lysine-proline-serine-tyrosine-proline-proline-threonine (SEQ ID NO: 14), and alanine-lysine-proline-serine-tyrosine-proline-proline-threonine (SEQ ID NO: 15)

9. The multicomponent adhesive as claimed in claim 1, wherein the first compound of the formula (I) has a structure according to the formula (IV)

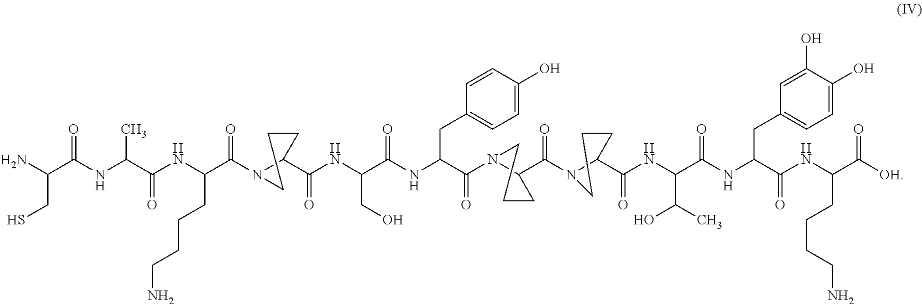 [SMILES: CC(NC(=O)C(N)CS)C(=O)NC(CCCCN)C(=O)N1CCCC1C(=O)NC(CO)C(=O)NC(Cc1ccc(O)cc1)C(=O)N1CCCC1C(=O)N1CCCC1C(=O)NC(C(=O)NC(Cc1ccc(O)c(O)c1)C(=O)NC(CCCCN)C(=O)O)C(C)O]

10. The multicomponent adhesive as claimed in claim 1, wherein the structural unit (II) has the following structure (IIa): 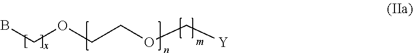
wherein
n is a whole number greater than or equal to 1,
x is selected from the group consisting of 0, 1, 2, 3, 4, 5, 6, 7, and 8,
and
m is selected from the group consisting of 0, 1, 2, 3, 4, 5, 6, 7, and 8.

11. The multicomponent adhesive as claimed in claim 1, wherein the second compound (b) comprises at least one compound selected from compounds of the formula (V) 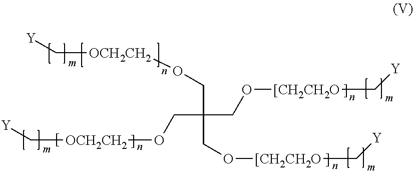
wherein
n in each of the four side chains independently of the meaning of n in the respective other side chains is a whole number greater than or equal to 1,
m in each of the four side chains independently of the meaning of m in the respective other side chains is selected from the group consisting of 0, 1, 2, 3, 4, 5, 6, 7, and 8,
and
Y in each of the four side chains independently of the meaning of Y in the respective other side chains is selected from the group consisting of SH, NH₂ and alpha,beta-unsaturated carbonyl groups and can be reacted with the group X with formation of at least one covalent bond.

12. The multicomponent adhesive as claimed in claim 1, wherein each of the structural units (III) has the following structure (IIIa): 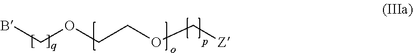
wherein
o is a whole number greater than or equal to 1,
q is selected from the group consisting of 0, 1, 2, 3, 4, 5, 6, 7, and 8,
and
p is selected from the group consisting of 0, 1, 2, 3, 4, 5, 6, 7, and 8.

13. The multicomponent adhesive as claimed in claim 1, wherein the third compound comprising the at last two structural units (III) comprises at least one compound selected from the group of compounds according to formula (VII) 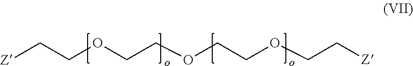
wherein o in each of the two side chains independently of the meaning of o in the respective other side chains is a whole number greater than or equal to 1, and
Z′ of each of the two side chains independently of the meaning of Z′ in the respective other side chains is selected from the group consisting of SH, NH₂ and alpha, beta-unsaturated carbonyl groups and can be reacted with a group Y with formation of at least one covalent bond.

14. The multicomponent adhesive as claimed in claim 1, configured for use in a method for surgical or therapeutic treatment of the human or animal body.

15. A method of muse of a multicomponent adhesive as claimed in claim 1 comprising non-therapeutic gluing of two surfaces.

16. A method for the production of an adhesive hydrogel comprising the following steps:
providing a multicomponent adhesive as claimed in claim 1, and
allowing the compounds of the multicomponent adhesive to react, so that the adhesive hydrogel is formed.

17. A kit comprising a multicomponent adhesive as claimed in claim 1 and at least one further component.

18. The kit as claimed in claim 17, wherein the further component is selected from the group consisting of syringes, cannulas, implants, stents, catheters and oxidizing agents for crosslinking an adhesive hydrogel produced from the multicomponent adhesive.

19. A gel comprising the multicomponent adhesive as claimed in claim 1,
wherein the gel is an adhesive hydrogel.

20. A gel comprising the multicomponent adhesive of claim 1,
wherein the gel is an adhesive hydrogel formed from a chemical reaction of the compounds of the multicomponent adhesive.
